(19) United States
(12) Patent Application Publication
(10) Pub. No.: US 2008/0220065 A1
(43) Pub. Date: Sep. 11, 2008

(54) Compositions for treating foot odor and methods of use thereof

(76) Inventor: David Robert Gelvin, Marietta, GA (US)

Correspondence Address: MYERS & KAPLAN;INTELLECTUAL PROPERTY LAW, L.L.C., CUMBERLAND CENTER II, 3100 CUMBERLAND BLVD , SUITE 1400, ATLANTA, GA, 30339, US

(21) Appl. No.: 11/715,016

(22) Filed: Mar. 7, 2007

(51) Int. Cl. A61K 31/19; A61K 8/36

(52) U.S. Cl. 424/486; 424/484; 424/69; 514/557

(57) ABSTRACT

A composition and method of use thereof for treating foot odor caused by microbial organisms, wherein cupric polyatomic salt is utilized as the active ingredient, preferably in combination with a dispersal agent, and wherein healthy tissue is promoted and foot odor is eliminated.

### TECHNICAL FIELD

The present invention relates generally to mixtures and methods for treating unpleasant odors, and more specifically, to compositions of cupric polyatomic salt for treating foot odor and methods of use thereof.

### BACKGROUND

Foot odor is a widespread problem for men, women, and children. Individuals engaging in activities resulting in frequent or continually wet or damp feet and/or footwear, and individuals unable to practice good hygiene are at an increased risk for bromphydrosis, or malodorous feet. Further, participants in some occupations and past-times are more prone to conditions that encourage foot odor problems, such as landscapers, factory workers, chemical workers, boat hands, wilderness adventurers, military service personnel, and gardeners, boaters, triatheletes, and hunters.

Commonly available formulations, such as powders, sprays, creams, ointments, tinctures, aqueous and non-aqueous solutions, suspensions, and shoe inserts, typically strive to absorb, neutralize, mask or deodorize unpleasant foot odors. However, the microbial organisms responsible for foot odor are not only easily spread to others, they are difficult to eliminate, even with repeated applications of presently available treatments. That is, currently available formularies disadvantageously lack strong evidence of efficient, long-term efficacy.

Some treatments utilize talc or sodium bicarbonate to assist in moisture absorption, along with perfumery components to assist in covering up offensive odors. For example, foot sprays are available with sodium bicarbonate, SD-Alcohol, and fragrance, and both foot sprays and powders are available in compositions including menthol. Menthol is frequently utilized as an antipruritic, to reduce itching, as well as in a perfumery capacity. Shoe insoles are offered with similar formulary, but provided via a matrix impregnated with sodium bicarbonate and fragrance components, amongst others.

Foot powders are also available with compositions containing anti-fungal, anti-bacterial keratolytic agents, primarily salicylic acid, anti-fungal, anti-bacterial agents, such as methyl salicylate, the methyl ester of salicylic acid, and moisture absorbing, dispersal agents, such as talc. Keratolytic agents, such as salicylic acid, are peeling agents that encourage the removal of dead skin cells. Methyl salicylate is a rubefacient that produces redness of the skin upon external application, by dilating capillaries and increasing blood circulation.

Thus, known compositions frequently contain acids and alcohols capable of inducing disadvantageous reactions, such as contact dermatitis, if topical administration is chronic or in sufficiently high concentrations. Further, salicylic acid and methyl salicylate are typically recognized as possessing, at most, only mildly effective anti-fungal, anti-bacterial capacity, and the available compositions thereof may be disadvantageously ineffectual in delivering the active agents to the site of contamination, and thus may be lacking in proven therapeutic value.

The effectiveness and long-term efficacy of prior art compositions may be dubious. A need therefore exists for a composition capable of overcoming the limitations of the prior art by delivering an appropriate non-toxic, persistent, heat and moisture stable agent at an effectual level to the source of the offending microbial organism responsible for foot odor, wherein extraneous delivery can result in active, anti-infective functionality with minimized risk of contact dermatitis.

### BRIEF SUMMARY OF THE INVENTION

Briefly described, in a preferred embodiment, the present invention overcomes the above-mentioned limitations and meets the recognized need by providing chemical compositions for treating foot odor and methods of use thereof with cupric polyatomic salt as an active ingredient, preferably cupric carbonate salt, CuCO₃—Cu(OH)₂, and preferably in conjunction with a dispersal agent, for preferred application in the treatment of microbe contaminated footwear, especially footwear harboring foul or unpleasant odors.

According to its major aspects and broadly stated, the present invention in its preferred form is a composition and method of administration thereof for preventing and eliminating foot odor caused by microbial organisms frequently inhabiting footwear, wherein cupric polyatomic salt is utilized to promote healthy tissue and to essentially eliminate the discomfort and embarrassment of foot odor via reduction of microbial contamination by administration of the composition to the foot, socks, an insertable matrix, such as an insole, or by direct application to footwear, wherein the composition is ideally suited to the ambient conditions of typical footwear.

More specifically, the preferred embodiment of the present invention comprises a mixture containing a biologically effective, therapeutic, non-toxic, persistent, stable quantity of an anti-infective agent, preferably in a mixture with a finely divided powder dispersal agent. Preferably, the anti-infective agent is cupric carbonate, CuCO₃—Cu(OH)₂, preferably at least one gram, in a quantity from approximately 35-60 wt/wt %, and preferably, the powder dispersal agent is ground magnesium silicate, i.e. talcum powder, though other powders having similar physical and chemical properties as talcum powder may be used and are considered to be within the scope of the present invention. Although unnecessary for realization of the anti-infective capability of the active agent, the inclusion of a dispersal agent indirectly increases effectiveness of the present invention in powdered or slurry form, when in the preferred composition from about 35-60 weight percent or 80-90 percent by volume, respectively. Further, various other polyatomic ions could be utilized in combination with, or as a replacement for the carbonate salt.

In lieu of or in addition to a finely divided powder dispersal agent, the cupric salt of the present invention may be mixed with an aqueous solvent as the dispersal agent to form a slurry. In such a composition, it is preferred that the cupric salt is at least one gram and is present in an amount approximately 10-20% volume of the mixture, and that the aqueous solvent is distilled water, though other aqueous solvents with physical and chemical properties similar to distilled water may be used and are considered to be within the scope of the present invention.

Additionally, in lieu of or in addition to the foregoing compositions and embodiments, the cupric salt of the present invention may be applied via an insole. In such an application, a disposable, multiuse, multilayer laminate shoe insert is preferred, comprising a plurality of layers of preferably polyatomic cupric salt coated fibers in a woven fabric, alternating with non-coated, preferably polyamide or polymer and natural fibers, wherein each layer containing a variable ratio of coated to uncoated fibers is preferably joined to adjacent layers. Similarly, fabric could be manufactured, infused with polyatomic cupric salt, wherein, for example, work boots or shoes could be manufactured therefrom with inherent odor-resistive qualities.

Accordingly, a feature and advantage of the present invention is its ability to provide a mixture of cupric polyatomic salt that treats foot-odor-causing microbes.

Another feature and advantage of the present invention is its ability to treat and decontaminate footwear contaminated by odor producing microbial organisms.

Another feature and advantage of the present invention is its ability to provide treated fabrics and materials for the manufacture of footwear that is resistant to contamination by odor producing microbial organisms.

Another feature and advantage of the present invention is its effectiveness and long-term efficacy relative to that of prior art compositions.

Still another feature and advantage of the present invention is the ability of the cupric polyatomic salt composition to be delivered at an effectual level to the source of the offending microbial organism(s) responsible for foot odor, via a plurality of non-toxic, persistent, heat and moisture stable agents.

Yet another feature and advantage of the present invention is its ability to provide active, anti-infective functionality with minimized risk of contact dermatitis.

Still yet another feature and advantage of the present invention is its ability to utilize cupric polyatomic salt to promote healthy tissue.

Yet still another feature and advantage of the present invention is its ability to essentially eliminate the discomfort and embarrassment of foot odor via reduction of microbial contamination by administration of the cupric polyatomic salt composition to the foot, socks, an insertable matrix, such as an insole, a constructive material, or by direct application to footwear, wherein the composition is ideally suited to the ambient conditions of typical footwear.

Another feature and advantage of the present invention is its ability to provide a mixture containing a biologically effective, therapeutic, non-toxic, persistent, stable quantity of cupric polyatomic salt as an anti-infective agent, either alone or in a mixture with a finely divided powder dispersal agent.

Still another feature and advantage of the present invention is its ability to disperse a composition of cupric atomic salt in an effective powdered or slurry form.

Yet still another feature and advantage of the present invention is its ability to utilize any combination of a plurality of polyatomic ions in combination with the divalent cupric ion.

Still yet another feature and advantage of the present invention is its ability to utilize an aqueous solvent as a dispersal agent for the active cupric polyatomic salt.

Still another feature and advantage of the present invention is its ability to apply the cupric salt via an insole.

These and other objects, features, and advantages of the invention will become more apparent to those ordinarily skilled in the art after reading the following Detailed Description of the Invention and Claims and the accompanying drawings.

### BRIEF DESCRIPTION OF THE DRAWINGS

The present invention will be better understood by reading the Detailed Description of the Preferred and Alternate Embodiments with reference to the accompanying drawing figures, in which like reference numerals denote similar structure and refer to like elements throughout, and in which:

FIG. 1 is a perspective view of an insole, according to a preferred embodiment of the present invention;

FIG. 2 is an exploded view of the insole of FIG. 1; and

FIG. 3 is an expanded, cutaway side view of the insole of FIG. 1.

### DETAILED DESCRIPTION OF THE INVENTION

In describing preferred embodiments of the present invention, specific terminology is employed for the sake of clarity. The invention, however, is not intended to be limited to the specific terminology so selected, and it is to be understood that each specific element includes all technical equivalents that operate in a similar manner to accomplish a similar purpose.

The present invention describes compositions for treating foot odor, and methods of use thereof, vis-a-vis the application of the compositions of present invention to typical and atypical footwear, either as applied directly to the footwear or via an impregnation of the present invention into shoe fabrication materials, insole inserts, and/or via application directly to the foot. The present invention also provides methods of preparing the compositions.

Preferably, the anti-infective compositions of the present invention contain a biologically active agent, presented in a biologically effective, therapeutic, persistent, non-toxic concentration, wherein the preferred agent is a polyatomic cupric salt. That is, the present invention provides compositions that are particularly effective at destroying and proactively inhibiting foot odor, wherein the active ingredient is a compound formed from copper (II) ions and associated salts.

Copper containing compositions have a long history of use as anti-infective agents, and are believed to upset microbial equilibrium, thereby interfering with maturation and reproduction of microbiotic organisms. For example, copper alloy surfaces are gaining in popularity for anti-microbial use, and copper fixtures are being recognized as offering proactive purification benefits not previously appreciated. It is believed that, based upon studies of cupric salts and their actions on mucus, namely human saliva, vaginal secretions, naso-pharyngeal and the excretory mucus production by common snails and slugs, that cupric salt disseminates the mucosal slime coat that certain bacteria employ as a primary line of defense, essentially turning the mucosal slime coat into water. It seems likely, then, that this defensive breech results in the organisms succumbing to the natural immune response of the host.

Investigative development of the preferred embodiment of the present invention was first focused toward the confirmatory establishment of the basic antimicrobial activities of cupric compounds, then to the identification of the preferred, active ingredient, the polyatomic cupric salt, and thereafter, was targeted to defining optimum concentrations with maximized anti-infective benefit, and to most efficacious and favorable modes of application.

### Anti-Infective Agent

Initial comparative studies were conducted in an effort to validate the antimicrobial activities of cupric compounds, wherein ubiquitous microbial cultures were collected from common household and mammalian sources and isolated in identifiable colonies by streak inoculation. Substrate filter discs were impregnated by aqueous saturation in either a carbonate slurry or acetate solution, and were subsequently incubated with the cultures, confirming the existence of antimicrobial properties in the cupric compounds.

Initial empirical use studies involved finely divided elemental copper powder. Use of such powder within a shoe, however, disadvantageously led to tissue and footwear damage via the formation of insoluble and sharp crystals from the reducible form of copper and the excreted electrolytes in sweat. Polyatomic salt studies followed, involving nitrate, chloride, and sulphate. Each, however, disadvantageously produced varying degrees of contact dermatitis, and was thus eliminated from consideration for further development.

Neutralization of cupric nitrate with sodium bicarbonate in an aqueous solution followed, with introduction into footwear accomplished via pouring approximately fifteen milliliters of the aqueous slurry onto the sole, tilting until dispersed about the interior shoe surface, and then decanting the excess. Although odor relief was realized, the process resulted in the deposit of surplus composition on the shoe interior, with disadvantageous side effects including excessive sock deposition, non-aesthetically appealing “bleed-through” at footwear seams, and mild contact dermatitis. The positive results with respect to the efficacy of the cupric carbonate product in controlling odor, however, led to further evaluative testing of lab grade cupric carbonate. Advantageously, contact dermatitis was completely eliminated when cupric carbonate was utilized.

Thus, a polyatomic cupric salt is the preferred active agent of the present invention, such as, for example, cupric carbonate, cupric acetate, cupric citrate, and/or cupric potassium chloride. Polyatomic cupric salts not listed above may be as or more effective than cupric carbonate, but are not preferably used because of perceived hazardous potential and possibly adverse dermatitic reactions. Such cupric salts include, but are not limited to: cupric bromide, cupric chloride, cupric fluroborate, cupric hydroxide, cupric nitrate, cupric oxide, and cupric sulfide.

The preferred polyatomic cupric salt active agent of the present invention is cupric carbonate, CuCO₃.Cu(OH)₂. Presently known uses of cupric carbonate include use as a coloring agent, and most commonly, in combination with arsenic to form the wood preservative and insecticide, PARIS GREEN. An appropriate raw material for the production of cupric carbonate is copper sulphate, the most widely used copper salt, and a raw material that is antiseptic and germicidally beneficial itself. Thus, one preferred method for the preparation of cupric carbonate is the addition of sodium carbonate, Na₂CO₃, to a solution of copper sulfate, CuSO₄, most frequently the pentahydrate, CuSO₄·5H₂O, for dehydration, filtering and drying, to produce the desired cupric carbonate. It should be recognized that any suitable method could be utilized.

In a preferred embodiment of the present invention, the concentration of the polyatomic cupric salt compound present in anti-infective compositions ranges from about 35-65 weight percent (wt/wt %), more preferably from about 40-60 wt/wt %, and even more preferably from about 45-55 wt/wt %, with a preferred minimum quantity of approximately one gram of active ingredient for realization of anti-infective properties.

Dosing specification studies were conducted in order to define a preferred minimum quantity for preferred use; although mindful of the differences in performance, more or less than the preferred minimum quantity could be utilized in alternate compositions. Initial test dosing was approximately 3.5 to 4.0 grams dispersed within each shoe, with excess bumped out through the footwear opening. Throughout a series of tests, the dosing quantity was successively reduced at intervals of 0.5 grams until therapeutic efficacy diminished.

In the study, six volunteer participants with confirmed and self-recognized foot odor participated in trials to establish preferred upper and lower specification ranges. Because earlier testing had already shown the efficacy of 4.0 gram dosing, this quantity served as the positive control for comparative odor analysis and was applied into the right shoe of each volunteer. The left shoes were treated in a variable fashion, from an initial 3.5 gram dose to a final dose of 0.5 grams, via 0.5 gram increments. An organoleptic evaluation was performed by each test subject, and also be the inventor, after three days, 10 days, and 30 days of treatment for each dose.

Final dosing results provided well-defined, consistent data, wherein all subjects dosed at or above 1.5 grams exhibited no detectable trace of odor beyond that of damp footwear. Additionally, and also consistently, all subjects dosed at or below 1.0 gram exhibited some malodorous relief, but for a more limited period of time, wherein foot odor gradually returned to pre-dosing levels.

Thereafter, to test the efficacy duration of the preferred mixture and concentration of the present invention, a study was conducted of twenty-six persons, male and female, ranging in ages from 7-71 years, all presenting with foul and distressing foot odor. These twenty-six persons had all of their “right-side” footwear treated with 1-1.25 g of cupric carbonate and their “left-side” footwear with 1-1.25 g sodium bicarbonate. Three days following administration, each shoe worn and both feet of each participant were evaluated via olfactory sense. Evaluation results were the same, without exception, for all participants. Namely, the “left-side” footwear and left feet treated with sodium bicarbonate demonstrated no obvious change in degree of unpleasant odor. All “right-side” footwear and right feet treated with cupric carbonate exhibited no detectable hint of unpleasantness, empirically proving the efficacy of the present invention. The trials showed the preferred compound to be nearly 100% effective for at least six months, and in many cases, twelve months or more.

Further, the anti-infective properties of the preferred composition of the present invention appear to be increased by heat and moisture, wherein desired functionality relative to shoes and shoe-enclosed feet is productively enhanced.

### Manufacture

In a preferred embodiment in which the present invention is comprised of a powdery mixture, the anti-infective composition of the present invention is preferably mixed with a dispersal agent such as a finely divided solid, for example, ground talc, though other powders having similar physical and chemical properties as talcum powder may be used and are considered to be within the scope of the present invention. By way of example, and not of limitation, inorganic and organic powders that may be utilized may include talc, kaolin, silica, mica, sericite, dolomite, phlogopite, lepidolite, biotite, vermiculite, magnesium carbonate, calcium carbonate, aluminum silicate, barium silicate, calcium silicate, magnesium silicate, strontium silicate, tungstic acid metal salts, magnesium, silica, zeolite, barium sulfate, sintered calcium sulfate, sintered gypsum, calcium phosphate, fluoroapatite, hydroxyapatite, ceramic powder, metal soap (zinc myristate, calcium palmitate, aluminum stearate), boron nitride, or any other suitable inorganic powder, and/or polyamide resin powders (nylon powders), polyethylene powder, polymethyl methacrylate powder, polystyrene powder, styrene-acrylate copolymer resin powder, benzoguanamine resin powder, polytetrafluoroethylene powder, cellulose powder, calcium alginate powder, or any other suitable organic powder.

As referenced in Table II, the weight percent of the finely divided solid in the mixture of the present invention is preferably in the range of approximately 35-65 wt/wt %, more preferably approximately 40-60 wt/wt %, and even more preferably approximately 45-55 wt/wt %, but mindful of the dose weight preferences established for the preferred active ingredient, cupric carbonate.

TABLE I

| Mode of | | | |
|---|---|---|---|
| Application of | | | |
| Polyatomic | | | |
| Salt | Range | Preferred | Best |
| Powdery | 35–65 wt/wt % | 40–60 wt/wt % | 45–55 wt/wt % |
| Mixture | finely | finely | finely |
| | divided | divided | divided |
| | solid | solid | solid |
| Slurry | 70–90 vol/vol % | 75–85 vol/vol % | 78–82 vol/vol % |
| | aqueous | aqueous | aqueous |
| | solvent | solvent | solvent |
| Powdery | 35–65 wt/wt % | 40–60 wt/wt % | 45–55 wt/wt % |
| Mixture + | finely | finely | finely |
| Additional | divided | divided | divided |
| Dispersal | solid + added | solid + added | solid + added |
| Agent(s) | dispersal | dispersal | dispersal |
| | agent | agent | agent |

In another embodiment, the present invention is a slurry comprised of the anti-infective cupric salt of the present invention mixed with an aqueous solvent. In this embodiment of the present invention, the aqueous solvent is preferably distilled water, though other aqueous solvents with physical and chemical properties similar to distilled water may be used and are considered to be within the scope of the present invention.

Preferably, the aqueous slurry mixture of the present invention is prepared with an aqueous solvent, such as distilled water, having a volume percentage of the volume mixture approximately 70-90 vol/vol %, more preferably approximately 75-85 vol/vol %, and even more preferably approximately 78-82 vol/vol %, again mindful of the dose weight preferences established for the preferred active ingredient, cupric carbonate. The anti-infective aqueous slurry mixture is preferably solubilized with freshly distilled water. Deionized water is not preferred because the deionizing resins can result in the presence of pathogens in the deionized water. Further, salts possibly present in the deionized water may react with and affect the polyatomic ion, thereby influencing the efficacy of the cupric salt of the present invention.

The aqueous anti-infective compositions may optionally include stabilizers and thickening agents to achieve viscosities within a useful range appropriate for the mode of application. Examples of stabilizers and thickening agents may include silica gel and emulsifying waxes. Other exemplary stabilizers and thickening agents, without limitation, include hydroxypropyl methylcellulose, hydroxypropyl cellulose, methyl cellulose, carboxy methylcellulose, emulsifying waxes, alkyl triammonium methosulfate, and ceteraryl octanoate, wherein exemplary waxes, without limitation, include beeswax, candellia wax, cotton wax, carnauba wax, bayberry wax, insect wax, spermaceti, rice bran wax, lanolin (all forms), kapok wax, sugar cane wax, hexyl laurate, jojoba wax, and shellac wax.

In another alternate embodiment of the present invention, additional dispersal agents may be employed, either in conjunction with or in place of the dispersal agents described hereinabove. Examples of such additional dispersal agents include, but are not limited to: tricalcium phosphate and cornstarch. Individually, it is preferred that total weight percentage of the mixture weight of the dispersal agents be present in the anti-infective mixture in essentially the same weight percentage ranges of the finely divided powder of the preferred embodiment of the present invention. In other words, preferably the total weight percentage of the dispersal agents is approximately 35-65 wt/wt %, more preferably approximately 40-60 wt/wt %, and even more preferably approximately 45-55 wt/wt %, again mindful of weight preferences empirically established for the preferred active ingredient, cupric carbonate.

The anti-infective compositions are generally prepared by blending the constituents together until an intimate mixture results, wherein the dispersal agents can also function to advantageously enhance the product application experience for the user. It is specifically noted, however, that the polyatomic salt of the present invention could be utilized without a dispersal agent. Nonetheless, preferably, an anti-caking agent or mixture thereof is present in the compositions. Further, if the present invention is prepared in the form of a slurry, thickening agents are preferably added proximate to the commencement of the mixing procedure. Mixture temperature may require adjustment to fully solubilize stabilizer agents. Other adjuvants, such as pH adjustors, can be blended with the compositions, as necessary, along with preservatives. Initial data indicates shelf life of the preferred composition may be extended relative to the extent of wear of the treated shoe, with preferred re-treatment occurring at approximately six months to one year, and with extended wear in wet or submerged conditions, preferred re-treatment occurring thereafter.

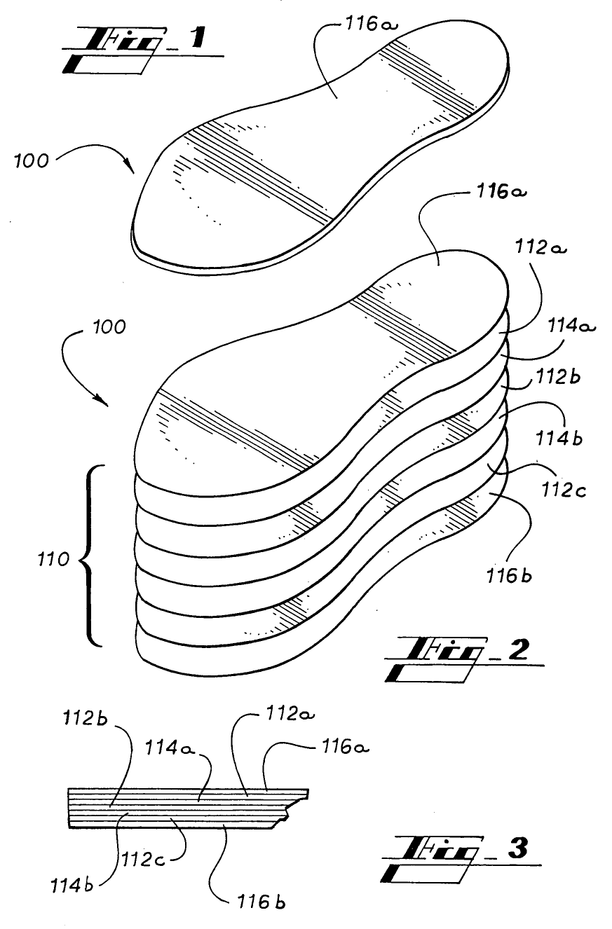

Another embodiment of the present invention comprises a coated/uncoated matrix of fibrous material for contact with the foot or shoe. Preferably, the matrix has alternating layers of synthetic and natural fibers, wherein preferably, the natural fibers are impregnated with the preferred polyatomic cupric salt mixture of the present invention. Referring to FIGS. 1-3, insole 100 is one embodiment of an impregnated matrix for delivery of the polyatomic cupric salt composition of the present invention, preferably including plurality of fibrous layers 110.

Plurality of fibrous layers 110 preferably comprise non-coated fibers 112a-112c and coated fibers 114a and 114b, wherein coated fibers 114a and 114b are at least partially coated with polyatomic cupric salt, preferably cupric carbonate. Fiber material utilized for coating with the mixture of the polyatomic cupric salt of the present invention preferably includes, without limitation, natural fibers, cotton, wool, silk, hemp, or other desirable fibers, natural or synthetic. Fiber material utilized for surrounding and essentially sandwiching preferred natural fibers 112a-c and 114a-b preferably defines finishing surfaces 116a and 116b, and preferably includes, without limitation, synthetic fibers,. rayon, nylon, g.lasswool, acrylic, thermoplastic polymers, or other desirable fiber, synthetic or natural.

Preferably, adjacent layers are joined together, wherein a preferably directly proportional ratio of coated to non-coated fibers define finished insole 100. Presumably, the preferred alternating of uncoated natural fiber layers 112a-c with coated natural fibers layers 114a-b facilitates laminate adhesion. Assembly preferably commences with fibrous sheets being submerged within or “run-through” a tank of agitated cupric carbonate slurry, and preferably continues with drying of same by artificial means. Additionally, but optionally, a copolymer may be introduced to enhance adhesion to natural fibers, wherein such introduction could be within the slurry or via direct coating of the fibrous sheets.

Adjacent layers of coated and non-coated fibers may also be joined together to form a woven fabric, finished with at least one surface of a durable permeable layer, such as, for exemplary purposes, a polymer or polyamide. Usefulness of the cupric polyatomic salt composition of the present invention, as described in the impregnated matrix of a woven fabric, in manufacture and construction of shoes and boots resistant to foot odor, is appreciated. Although use is not limited, one particularly apparent application of such an embodiment is the manufacture of dedicated shoes and boots for particular professions and activities frequently subject to wet and undesirable conditions, such as firefighters, military personnel, athletes, and hikers.

### Application

The anti-infective mixture of the present invention can be applied in various conventional forms, including: cream, salve, powder, suspension, aerosol, swab, mist, gel, paste, lotion, ointment, and impregnated matrix. Administration of the composition of the present invention preferably comprises the step of applying an adequate quantity of the cupric polyatomic salt mixture of the present invention to one's feet, socks, and/or footwear, to cover the entire afflicted area. The application is preferably made about once per year, or as circumstances otherwise require, such as when new, untreated footwear is acquired.

Application of the preferred powdery mixture with a finely divided solid is preferably accomplished by introduction into a selected article of footwear, wherein equal distribution is preferably accomplished by alternately striking the heel and toe of the footwear on a solid surface. Application of the slurried mixture is preferably accomplished via introduction into a selected article of footwear, wherein equal distribution is preferably accomplished by alternately tilting between heal and toe of the footwear.

Application via a coated/uncoated matrix in the form of a shoe insole insert is also intended within the scope of the present invention. Further, application of the polyatomic cupric salt composition of the present invention is suitable for application to open-toe footwear, sandals, disposable footwear, such as booties and pedicure slippers, and specialty-type footwear, such as water socks and scuba diving boots.

Additionally, the anti-infective compositions of the present invention are also suitable for use as cleansing compositions for impermeable footwear, such as, for example, rubber boots and fishing waders. Further, the compositions can be used in a prophylactic manner for shared footwear such as rented bowling shoes, and ice and roller skates.

Having, thus, described exemplary embodiments of the present invention, it should be noted by those skilled in the art that the within disclosures are exemplary only and that various other alternatives, adaptations, and modifications may be made within the scope and spirit of the present invention. Accordingly, the present invention is not limited to the specific embodiments as illustrated herein, but is only limited by the following claims.

I claim:

1) A fine solid composition for treating foot odor, comprising:
cupric carbonate present in about 40-60 WT %; and
a dispersal agent comprising a finely divided inert powder present in about 40-60 WT %.

2) A slurry composition for treating foot odor, comprising:
cupric carbonate present in about 10-30 wt %; and
an aqueous solvent dispersal agent comprising distilled water present in about 70-90 WT %.

3) An impregnated matrix for treating foot odor, comprising:
a plurality of layers of a fibrous material, wherein at least some portion of said fibrous material is coated with cupric carbonate.

4) The impregnated matrix of claim 3, wherein said plurality of layers are joined and wherein the ratio of said coated to non-coated fibers is proportional.

5) The impregnated matrix of claim 4, wherein said plurality of joined layers comprises at least one outer, durable, permeable layer.

6) The impregnated matrix of claim 5, wherein said at least one outer, durable, permeable layer is comprised of a polyamide.

7) The impregnated matrix of claim 5, wherein said at least one outer, durable, permeable layer is comprised of a polymer.

8) The impregnated matrix of claim 3, wherein at least a portion of said fibrous material is selected from the group of natural fibers comprising: cotton, wool, silk, or hemp.

9) The impregnated matrix of claim 3, wherein at least a portion of said fibrous material is selected from the group of synthetic fibers comprising: rayon, nylon, glasswool, acrylic and thermoplastic polymers.

10) A mixture for treating foot odor, comprising:
polyatomic cupric salt; and
a first dispersal agent.

11. The mixture of claim 10, wherein said first dispersal agent is a finely divided solid.

12. The mixture of claim 11, wherein said finely divided solid is selected from the group comprising: talc, kaolin, silica, mica, sericite, dolomite, phlogopite, lepidolite, biotite, vermiculite, magnesium carbonate, calcium carbonate, aluminum silicate, barium silicate, calcium silicate, magnesium silicate, strontium silicate, tungstic acid metal salts, magnesium, silica, zeolite, barium sulfate, sintered calcium sulfate, sintered gypsum, calcium phosphate, fluoroapatite, hydroxyapatite, ceramic powder, zinc myristate, calcium palmitate, aluminum stearate, boron nitride, polyamide resin powders, nylon powders, polyethylene powder, polymethyl methacrylate powder, polystyrene powder, styrene-acrylate copolymer resin powder, benzoguanamine resin powder, polytetrafluoroethylene powder, cellulose powder, or calcium alginate powder.

13. The mixture of claim 11, wherein said finely divided solid is present in the mixture in a range of about 35% to about 65% by weight.

14. The mixture of claim 11, wherein said finely divided solid is present in the mixture in a range of about 40% to about 60% by weight.

15. The mixture of claim 11, wherein said finely divided solid is present in the mixture in a range of about 45% to about 55% by weight.

16. The mixture of claim 10, wherein said first dispersal agent is an aqueous solvent.

17. The mixture of claim 16, wherein said aqueous solvent is distilled water.

18. The mixture of claim 17, wherein said distilled water is present in the mixture in a range of about 70% to about 90% by volume.

19. The mixture of claim 17, wherein said distilled water is present in the mixture in a range-of about 75% to about 85% by volume.

20. The mixture of claim 17, wherein said distilled water is present in the mixture in a range of about 78% to about 82% by volume.

21. The mixture of claim 10 further comprising a viscosity modification agent.

22. The mixture of claim 10, further comprising a second dispersal agent.

23. The mixture of claim 10, wherein said mixture is impregnated into a fibrous matrix.

24. A method for treating foot odor, comprising the steps of:
a) obtaining a composition comprising polyatomic cupric carbonate; and
b) applying said composition to a shoe.

25. The method of treating foot odor of claim 24, wherein said composition is applied to a foot in lieu of a shoe.

26. The method for treating foot odor of claim 24, wherein said composition is impregnated within a matrix, and wherein said matrix is placed in substantial contact with an odorous source.

27. The method for treating foot odor of claim 24, wherein said composition further comprises a dispensing agent.
